(19) United States
(12) Patent Application Publication
(10) Pub. No.: US 2022/0233132 A1
(43) Pub. Date: Jul. 28, 2022

(54) DEVICE FOR THE TREATMENT OF DYSTONIA

(71) Applicant: University of South Florida, Tampa, FL (US)

(72) Inventors: Stephanie Lutton Carey, Tampa, FL (US); Arsany Boles Gad, Tampa, FL (US); Jordyn Elizabeth Bizzell, Tampa, FL (US); Juan Sanchez-Ramos, Tampa, FL (US); Sang-Hie Lee, Tampa, FL (US); Ying Zhong, Tampa, FL (US)

(21) Appl. No.: 17/582,396

(22) Filed: Jan. 24, 2022

Related U.S. Application Data
(60) Provisional application No. 63/140,505, filed on Jan. 22, 2021.

(51) Int. Cl. A61B 5/395; A61B 5/00; A61B 5/265

(52) U.S. Cl. CPC A61B 5/395; A61B 5/7455; A61B 5/0059; A61B 5/6806; A61B 5/265; A61B 5/742

(57) ABSTRACT

One or more EMG sensors are configured to sense surface-EMG data indicative of muscle movement of the patient; and transmit surface-EMG data. A controller can identify at least one parameter of the surface-EMG data; compare the parameter to a corresponding dystonia-threshold-value; responsive to a determination that the parameter is greater than the corresponding dystonia-threshold-value: issue a visual-engagement command to a visual unit; and issue a tactile-engagement command to a tactile unit.

### CROSS-REFERENCE TO RELATED APPLICATIONS

This application claims priority to U.S. Provisional Application No. 63/140,505, filed Jan. 22, 2021, the disclosure of which is incorporated herein by reference in its entirety.

### TECHNICAL FIELD

This document describes technology that can be used for the treatment of medical disorders such as Focal Dystonia which in some cases includes an oscillatory component resembling tremors.

### BACKGROUND

Dystonia is a neurological condition in which muscles contract involuntarily, causing repetitive, twisting movements and abnormal posturing. The condition can affect one part of the body (focal dystonia), two or more adjacent parts (segmental dystonia) or multiple segments of the body (general dystonia). Focal, task specific dystonia (FTSD) occurs in a specific area of the body and is triggered by repetitive movements, which in some cases may be a symptom of early onset Parkinson's Disease, etc. FTSD usually appears symptomatically after the fourth decade of life and is more common in men than in women. FTSD can affect writers, typists, musicians, athletes, and any other individuals that spend a large amount of time making repetitive movements. FTSD can have a negative impact on quality of life because of the motor tasks it affects.

FTSD has a high rate of incidence amongst the musician population. This subset of FTSD is often referred to as “musician's dystonia” (MD) and can manifest as hand or embouchure dystonia. FTSD also affects non-musicians in lesser numbers. Writer's cramp is the most common upper-limb form of FTSD and causes an uncomfortable stiffness that can make it difficult to write efficiently.

Electromyography (EMG) is defined as the electrical signal measured from muscle contractions. EMG can provide data on how much a muscle is being contracted, which can prove useful in detecting dystonic movements. EMG can be measured in two ways, surface and intramuscular. Surface EMG is usually used to measure superficial muscles, while intramuscular can measure deeper muscles.

### SUMMARY

Described in this document is technology for the therapy of dystonic medical disorders such as Focal Dystonia, Parkinson's Resting Tremors, and Essential Tremors. This technology uses device to provide additional sensory feedback in order to interrupt the sensorimotor pathways that cause dystonia. This can provide a solution that is organic, physical in nature providing the body with additional sensory information via vibrating tactors so that the focal dystonia is interrupted in a natural, biological way.

In one implementation, a system is used for the treatment of dystonic symptoms. The system includes one or more surface-electromyogram (EMG) sensors configured to: attach to a patient; sense surface-EMG data indicative of muscle movement of the patient; and transmit, to a controller, the surface-EMG data; and active treatment component, e.g. tactors. The system includes a controller comprising computer-memory and one or more processors, the computer memory storing instructions that, when executed by the processor, cause the controller to: receive, from the surface-EMG sensors, the surface-EMG data; identify at least one parameter of the surface-EMG data; compare the parameter to a corresponding dystonia-threshold-value; responsive to a determination that the parameter is greater than the corresponding dystonia-threshold-value: issue a sensory-engagement command to a visual unit; and issue a tactile-engagement command to a tactile unit. The system includes the unit configured to: receive the—engagement command; and responsive to receiving the—engagement command, generating a stimulation for the patient. The system includes the tactile unit configured to: receive the tactile-engagement command; and responsive to receiving the tactile-engagement command; generating a tactile stimulation for the patient. Other implementations include methods, devices, software, computer-readable media, and other systems for the treatment of dystonic symptoms.

Implementations can include some, all, or none of the following features. The system further comprises a harness shaped to be wearable by the patient, the harness further being configured to secure the surface-EMG sensors, the controller, the visual unit, and the tactile unit. The harness comprises a sleeve wearable on the patient's arm. The harness comprises a glove wearable on the patient's hand. The surface-EMG sensors comprise graphene and a silver electrode. The surface-EMG sensors are configured to be worn on the patient's forearm. The surface-EMG sensors are configured to sense surface-EMG data indicative of muscle movements in at least one of the group consisting of Flexor Carpi Ulnaris, Palmaris Longus, Extensor Digitorum, and Extensor Carpi Radialis. The surface-EMG sensors are configured to sense surface-EMG data indicative of muscle movements in each of the group consisting of Flexor Carpi Ulnaris, Palmaris Longus, Extensor Digitorum, and Extensor Carpi Radialis. At least one parameter is a dystonic signal present in the surface-EMG data when the patient is experiencing dystonic symptoms. The system is configured to deliver the stimulation to the patient when the patient is experiencing dystonic symptoms. The system is configured to deliver the tactile stimulation to the patient when the patient is experiencing dystonic symptoms. The system further comprising at least one battery in energetic communication with at least the controller. The stimulation comprises illumination of a light-emitting diode (LED). The tactile stimulation comprises engagement of a tactor that generates a vibration on the patient's skin. The parameter is a first value in volts and wherein the corresponding dystonia-threshold-value is a second value in volts.

Implementations can include some, all, or none of the following advantages. The treatment of conditions such as Focal Dystonia, Parkinson's Resting Tremors, and Essential Tremors can be improved. The technology can be used in patient treatment to prevent or limit the amount of discomfort and uncomfortable twisting postures experienced. The technology could be used to detect that these movements are going to occur before they happen and prevent the unintended action from interrupting important tasks like writing and typing. For professional musicians, it could allow them to play through their set without having to take a break or stopping in the middle of a performance. The technology can help prevent the unwanted and uncomfortable dystonic movements associated with task-induced focal dystonia. These movements are an issue for people who regularly type, write, or play an instrument, and this technology can improve such people's quality of life. This condition occurs famously in piano players, who need the complete and free usage of their hands in order to continuously play. Along with individuals who type and write regularly in their jobs, these groups of people need relief from the dystonic symptoms in order to continue work in their professions. Bringing relief to their symptoms with this technology allows them train and retrain with peace of mind and comfort when performing tasks that are a huge part of their life. This technology can be used in a drug-free therapy for such disorders, which may provide benefits such as the avoidance of drug-related side effects and may provide particularly beneficial relief to special populations that are not able to tolerate drug-based therapies.

### DESCRIPTION OF DRAWINGS

FIG. 1 shows a schematic diagram of a system for the treatment of dystonic symptoms.

FIG. 2 shows a component diagram of a system for the treatment of dystonic symptoms.

FIG. 3 shows locations of sensors on a human arm.

FIG. 4 shows a swimlane diagram of a process for the treatment of dystonic symptoms.

FIG. 5 is a schematic diagram that shows an example of a computing device and a mobile computing device.

FIG. 6 shows two examples of sensor hardware.

Like reference symbols in the various drawings indicate like elements

### DETAILED DESCRIPTION

This document describes technology that can be used to treat dystonic symptoms. Surface electromyogram (EMG) sensors can be used to sense muscle movements of a patient, and can transmit EMG data to a controller. The controller can monitor the EMG data to determine if the muscle movements include unwanted dystonia. If the dystonia is detected, the controller can issue commands to engage stimulation of the patient, for example in the form of visual stimulation (e.g., light emitting diodes or LED illumination) and/or tactile stimulation (e.g., vibrations of one or more tactors). When the patient receives this stimulus, the patient can be cued to engage in practices from neuroplasticity retraining, other volitional modalities. In addition or in the alternative, the stimulus may also execute so-called “sensory tricking” in which new stimulus allows the patient to ameliorate the dystonia non-volitionally or extra-volitionally.

In some cases, this technology can include surface EMGs (sEMGs) attached to selected muscles of the ventral and dorsal forearm using an elastic sleeve with embedded surface EMG electrodes will permit muscle activity. This arrangement can include a wireless bioamplifier system with 4-8 channels permitting continuous recording of the pattern of muscle activity of the forearm. The elastic sleeve containing the sEMGs may also have embedded a pattern of tactors over selected forearm muscles. These tactors are miniature vibrotactile transducer that has been configured to create a strong localized sensation on the site that it is placed. A body-referenced arrangement of tactors can be activated individually, sequentially or in groups. These tactors can be controlled with a multi-channel controller/interface board and software that can be programmed to delivery vibrotactile stimulation in response to sEMG signals above a specific threshold.

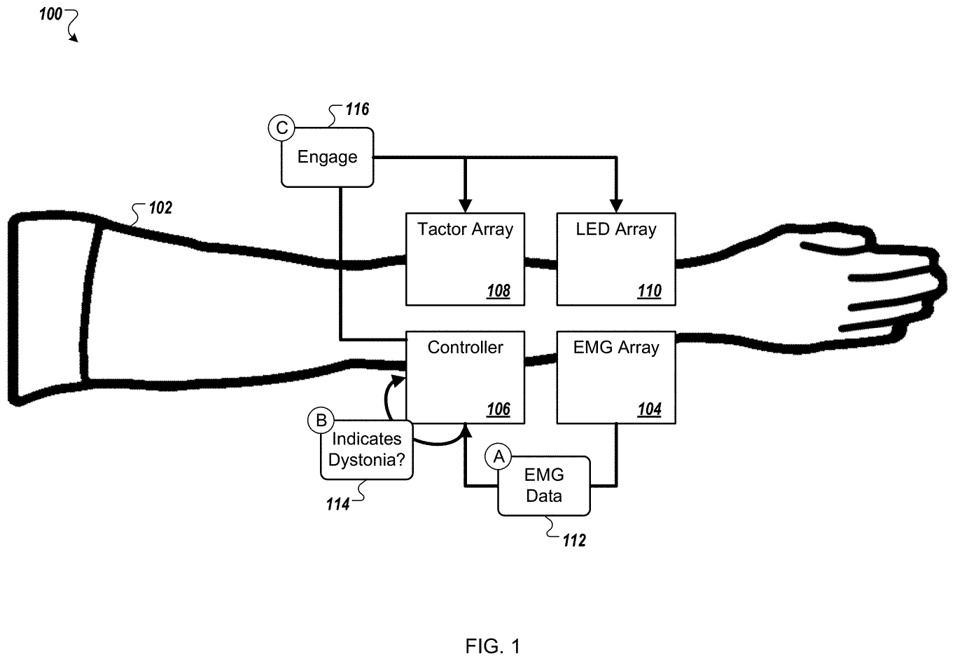

FIG. 1 shows a schematic diagram of a system 100 for the treatment of dystonic symptoms. The system 100 is configured to deliver the visual stimulation to the patient when the patient is experiencing dystonic symptoms. In the system 100, a patient is wearing a harness 102 that incorporates an array 104 of one or more EMG sensors. These EMG sensors provide data 112 to a controller 106 that can examine the data for signs of dystonia. If dystonia is detected 114, the controller can send commands 116 to stimulator elements such as a tractor array 108 to provide tactile stimulation to the user and an LED array 110 to provide visual stimulation to the user.

Said another way, the technology monitors the patient for dystonia symptoms such as repetitive, twisting movements and abnormal posturing. When those symptoms are sensed, the user is stimulated with stimulus that leads to volitional or non-volitional reduction in dystonia symptoms. To accomplish this effect, the system 100 is configured to deliver the visual stimulation to the patient when the patient is experiencing dystonic symptoms and the system 100 is configured to deliver the tactile stimulation to the patient when the patient is experiencing dystonic symptoms. This can result in the patient sending a signal to their brain when experiencing the dystonic symptoms.

The harness 102 shown her is a glove and sleeve worn on the patient's arm. This harness 102 can include one or more fabrics such as cotton, polyester, lycra, hook-and-loop, etc. The harness 102 may be shaped in such a way as to securely be worn on the arm during normal activities (e.g., typing, eating) and vigorous activities (e.g., playing musical instruments, exercising). The harness 102 can be formed to secure the elements 104-110 of the system 100. For example, one or more pockets, flaps, recesses, buttons, or zippers may be formed in the harness 102 to secure these elements 104-110. While a sleeve and glove are shown here, other forms may be used for wearing on portions of the body as is appropriate for a particular patient. For example, other harnesses may be only sleeves or only gloves, or may take the form of harnesses formed to be worn on feet, legs, the torso, a collar worn on the neck, etc.

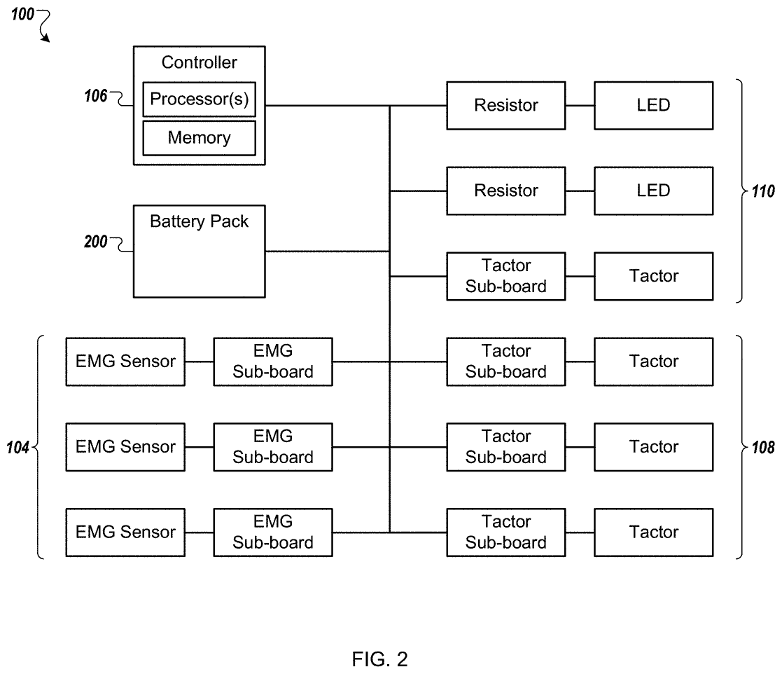

FIG. 2 shows a component diagram of the system 100 for the treatment of dystonic symptoms. Shown here, in addition to the elements 104-110, is a battery pack 200.

The EMG array 104 can include one or more EMG sensors and one or more EMG sub-boards. While three of each such components are shown here, it will be understood that more or fewer are possible, including different numbers of each (e.g., one sub-board servicing multiple EMG sensors). The EMG sensors may be formed to make contact with the surface of a patient to attach to the patient. One example EMG sensor is a graphene sensor with one or more silver electrode, though other configurations are possible, such as carbon nanotubes (CNT), other metals (copper), nanowire, nanoparticles, etc. The EMG sub-boards can include an interface to communicate with the controller 106 for the passage of data (e.g., via digital communication) and a second interface for connection with the EMG sensor (e.g., via analog communication). The EMG sub-boards can further include hardware, firmware, and/or software that is configured to turn the (e.g., analog) sensor readings into (e.g., digital) data for the controller. Such hardware can include an analog-to-digital converter, an amplifier, a filter, etc.

The controller 106 can include one or more processors and one or more computer memory modules, along with other hardware. The computer memory can store instructions that are executable by the processor(s) that cause the controller 106 to perform various actions including, but not limited to, receiving data from the EMG sensor array 106, perform computational operations on this data, and transmit data to the tractor array 108 and/or the LED array 110 (e.g., see the process 400 described below).

The LED array 110 can include one or more LEDs and hardware, firmware, and/or software configured to drive the LEDs to emit light, depending on received data, analog or digital. Shown here, the LEDs are connected to resistors and can receive an analog electrical signal from the controller 106, causing the LEDs to emit light. In another configuration, one or more LED sub-boards can include hardware, firmware, and/or software to receive digital instructions and to drive the LEDs based on digital instructions received from the controller. While three of each such components are shown here, it will be understood that more or fewer are possible, including different numbers of each (e.g., one resistor or sub-board servicing multiple LEDs).

The tractor array 108 can include one or more tractors and hardware, firmware, and/or software configured to drive the tractors to vibrate, depending on received data, analog or digital. In general, tractors are small actuators that vibrate against the skin to provide a physical stimulus in response to an electrical input. Shown here, the tractors are connected to corresponding sub-boards that can include an interface to communicate with the controller 106 for the passage of data (e.g., via digital communication) and a second interface for connection with the tractors (e.g., via analog communication). The tractor sub-boards can further include hardware, firmware, and/or software that is configured to turn the (e.g., digital) data from the controller into (e.g., analog) control signals for the tractors. Such hardware can include an analog-to-digital converter, an amplifier, a filter, etc. While three of each such components are shown here, it will be understood that more or fewer are possible, including different numbers of each (e.g., one sub-board servicing multiple tractors).

The battery pack 200 can include one or more batteries in energetic communication with elements of the system such as the controller 106 and the subboards. The batter pack 200 can include a housing for removable batters or may permanently house one or more rechargeable batteries.

Connections between the elements 104-110 and 200 of the system 100 can be accomplished through one or more network connections. These network connections may include wired communication links as well as wireless communication links.

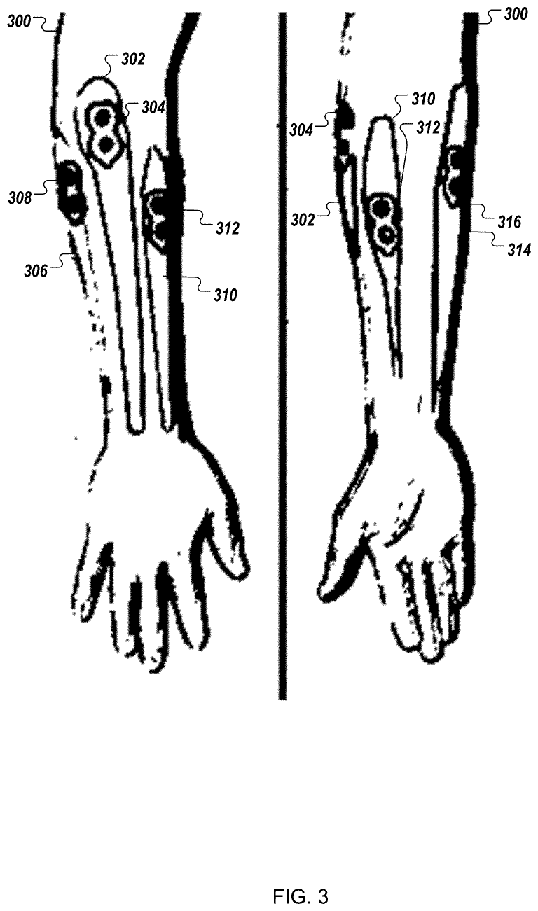

FIG. 3 shows locations of sensors on a human arm. For example, the EMG sensors in the array 104 may be worn on the forearm 300 of a patient in the arrangement shown.

In the area 302 of the Extensor Digitorum, EMG sensors 304 may be worn. In the area of the Extensor Carpi Radialis 306, EMG sensors 308 may be worn. In the area of the Palmaris Longus 310, EMG sensors 312 may be worn. In the area 314 of the Flexor Carpi Ulnaris, sensors 316 can be worn.

Various combinations of the sensors shown may be worn in different instances. For example, EMG sensors may be placed in every location shown here such that sensors are able to sense surface-EMG data indicative of muscle movements in each of the group consisting of Flexor Carpi Ulnaris, Palmaris Longus, Extensor Digitorum, and Extensor Carpi Radialis. In another example, one some of the sensors shown here may be placed, such that sensors are able to sense surface-EMG data indicative of muscle movements in at least one, but less than all of, of the group consisting of Flexor Carpi Ulnaris, Palmaris Longus, Extensor Digitorum, and Extensor Carpi Radialis.

As can be seen in the FIG. 3, the sensing area of the sensors can be configured to be smaller and/or more localized than the total area of the harness 102. As such, the sensing area of the harness 102 can be configured to be smaller than the area of the tactor array 108. Said another way, the sensing area can be smaller or more localized than the area of the body to which tactile stimulation can be provided. However, other alternatives are possible such as identical sensing and stimulation areas, equal-sized but different-shaped sensing and stimulation areas, and larger sensing areas than stimulation areas.

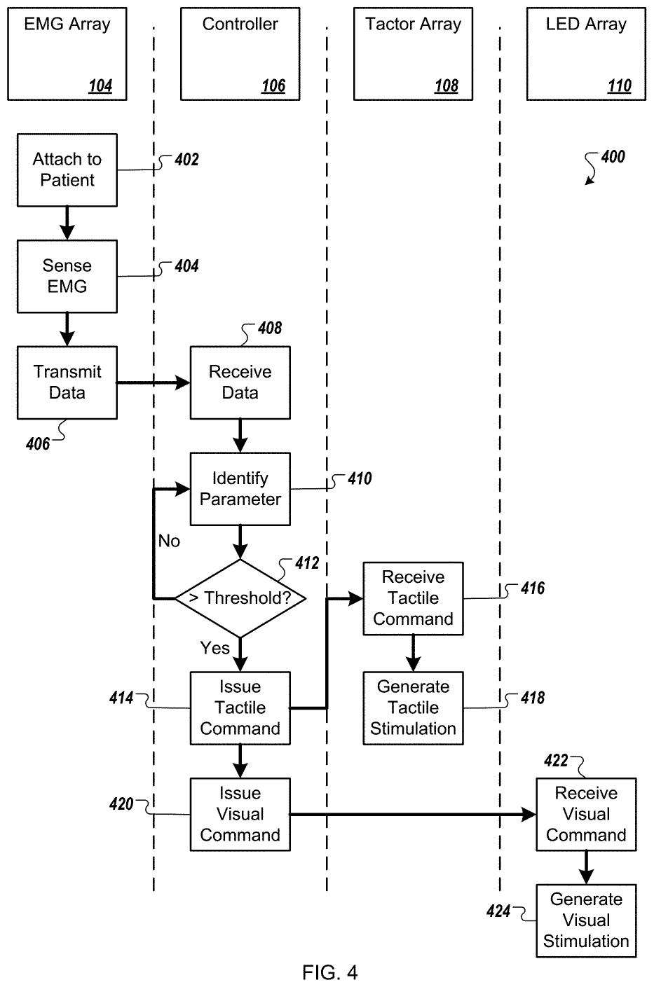

FIG. 4 shows a swimlane diagram of a process 400 for the treatment of dystonic symptoms. For example, the process 400 can be used with the system 100, and, as such, the system 100 will be used when discussing the process 400. However, another systems or systems may be used to perform the process 400 or other similar processes.

The EMG array 104 is attached 402 to a patient. For example, sensors can be placed on the patient in an area where the patient has experienced dystonia symptoms. Example arrangements of EMG sensors on a forearm are shown with respect to FIG. 3 above.

The EMG array 104 senses 404 surface-EMG data indicative of muscle movement of the patient. For example, as the patient voluntarily or involuntarily activates various muscles that are under the EMG sensors, the EMG sensors can sense changes in the electrical field in the patient's body. These changes can be transformed by the EMG sensors in to analog electrical signals accessible to the system 100.

The EMG array 104 transmits 406, to the controller 106, the surface-EMG data. The controller 106 receives 408, from the EMG array, the surface-EMG data. For example, the EMG array can convert these analog electrical signals into corresponding digital signals with values that represent the magnitude of the voltage (e.g., in the form of numeric representations of micro-voltage), resistance, etc. These digital values can be transmitted from the EMG array 104 to the controller 106 over a wired or wireless data connection.

The controller 106 identifies 410 at least one parameter of the surface-EMG data. For example, one parameter identified may be a voltage (e.g., micro-voltage) value that is indicative of the type, intensity, or periodicity of muscle activation by the patient. This can include voltage values produced when the patient is experiencing dystonic symptoms as well as voltage values from other types when the patient is not experiencing dystonic symptoms. In another example, resistance can be used instead of volts.

The controller 106 compares 410 the parameter to a corresponding dystonia-threshold-value. For example, the controller 106 can store, in computer memory, one or more numeric values representing micro-voltage values that indicate dystonic symptoms. These threshold values may be individualized for the particular patient, or may be generalized for a population. In some cases, different threshold values may be used for different sensors such as different sensors attached to different muscle groups. If the parameters are found to be less than the threshold value, the process 400 can repeat, looking for parameters greater than the threshold value.

Responsive to a determination 412 that the parameter is greater than the corresponding threshold-value, the controller 106 issues 414 a tactile-engagement command to the tractor array 108. The tractor array 108 can receive 416 the tactile command and generate 418 tactile stimulation. For example, this can represent the controller 106 identifying dystonic muscle contractions, and issuing commands to engage tactile stimulation in an effort to volitionally or non-volitionally reduce or remove the dystonic muscle contractions.

Responsive to a determination 410 that the parameter is greater than the corresponding threshold-value, the controller 106 issues 420 a visual-engagement command to the LED array 110. The LED array 110 can receive 422 the visual command 422 and generate 424 visual stimulation. For example, this can represent the controller 106 identifying dystonic muscle contractions, and issuing commands to engage visual stimulation in an effort to volitionally or non-volitionally reduce or remove the dystonic muscle contractions.

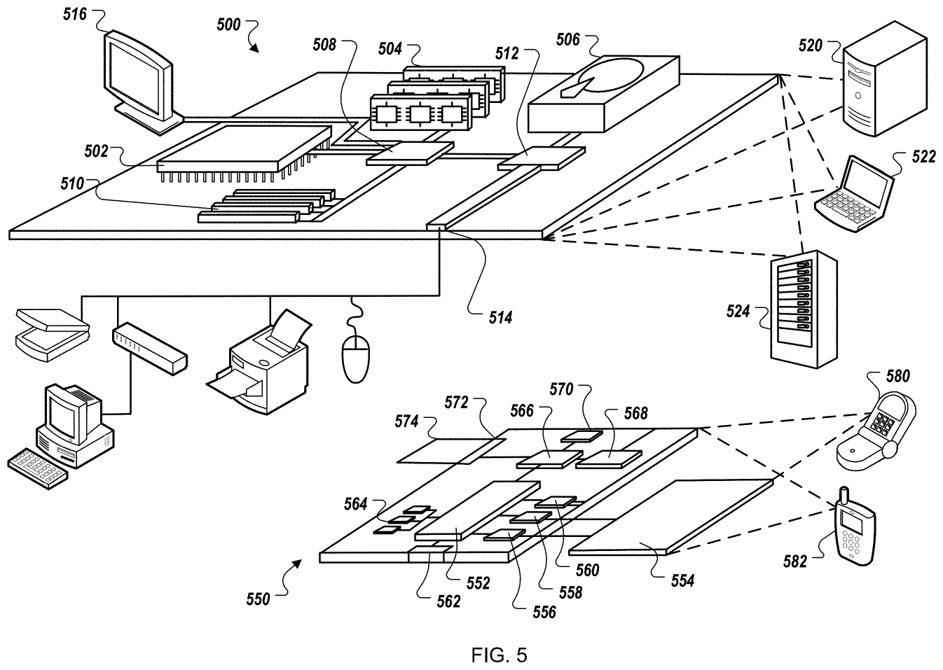

FIG. 5 shows an example of a computing device 500 and an example of a mobile computing device that can be used to implement the techniques described here. The computing device 500 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 500 includes a processor 502, a memory 504, a storage device 506, a high-speed interface 508 connecting to the memory 504 and multiple high-speed expansion ports 510, and a low-speed interface 512 connecting to a low-speed expansion port 514 and the storage device 506. Each of the processor 502, the memory 504, the storage device 506, the high-speed interface 508, the high-speed expansion ports 510, and the low-speed interface 512, are interconnected using various busses, and can be mounted on a common motherboard or in other manners as appropriate. The processor 502 can process instructions for execution within the computing device 500, including instructions stored in the memory 504 or on the storage device 506 to display graphical information for a GUI on an external input/output device, such as a display 516 coupled to the high-speed interface 508. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 504 stores information within the computing device 500. In some implementations, the memory 504 is a volatile memory unit or units. In some implementations, the memory 504 is a non-volatile memory unit or units. The memory 504 can also be another form of computer-readable medium, such as a magnetic or optical disk.

The storage device 506 is capable of providing mass storage for the computing device 500. In some implementations, the storage device 506 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 504, the storage device 506, or memory on the processor 502.

The high-speed interface 508 manages bandwidth-intensive operations for the computing device 500, while the low-speed interface 512 manages lower bandwidth-intensive operations. Such allocation of functions is exemplary only. In some implementations, the high-speed interface 508 is coupled to the memory 504, the display 516 (e.g., through a graphics processor or accelerator), and to the high-speed expansion ports 510, which can accept various expansion cards (not shown). In the implementation, the low-speed interface 512 is coupled to the storage device 506 and the low-speed expansion port 514. The low-speed expansion port 514, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 500 can be implemented in a number of different forms, as shown in the figure. For example, it can be implemented as a standard server 520, or multiple times in a group of such servers. In addition, it can be implemented in a personal computer such as a laptop computer 522. It can also be implemented as part of a rack server system 524. Alternatively, components from the computing device 500 can be combined with other components in a mobile device (not shown), such as a mobile computing device 550. Each of such devices can contain one or more of the computing device 500 and the mobile computing device 550, and an entire system can be made up of multiple computing devices communicating with each other.

The mobile computing device 550 includes a processor 552, a memory 564, an input/output device such as a display 554, a communication interface 566, and a transceiver 568, among other components. The mobile computing device 550 can also be provided with a storage device, such as a micro-drive or other device, to provide additional storage. Each of the processor 552, the memory 564, the display 554, the communication interface 566, and the transceiver 568, are interconnected using various buses, and several of the components can be mounted on a common motherboard or in other manners as appropriate.

The processor 552 can execute instructions within the mobile computing device 550, including instructions stored in the memory 564. The processor 552 can be implemented as a chipset of chips that include separate and multiple analog and digital processors. The processor 552 can provide, for example, for coordination of the other components of the mobile computing device 550, such as control of user interfaces, applications run by the mobile computing device 550, and wireless communication by the mobile computing device 550.

The processor 552 can communicate with a user through a control interface 558 and a display interface 556 coupled to the display 554. The display 554 can be, for example, a TFT (Thin-Film-Transistor Liquid Crystal Display) display or an OLED (Organic Light Emitting Diode) display, or other appropriate display technology. The display interface 556 can comprise appropriate circuitry for driving the display 554 to present graphical and other information to a user. The control interface 558 can receive commands from a user and convert them for submission to the processor 552. In addition, an external interface 562 can provide communication with the processor 552, so as to enable near area communication of the mobile computing device 550 with other devices. The external interface 562 can provide, for example, for wired communication in some implementations, or for wireless communication in other implementations, and multiple interfaces can also be used.

The memory 564 stores information within the mobile computing device 550. The memory 564 can be implemented as one or more of a computer-readable medium or media, a volatile memory unit or units, or a non-volatile memory unit or units. An expansion memory 574 can also be provided and connected to the mobile computing device 550 through an expansion interface 572, which can include, for example, a SIMM (Single In Line Memory Module) card interface. The expansion memory 574 can provide extra storage space for the mobile computing device 550, or can also store applications or other information for the mobile computing device 550. Specifically, the expansion memory 574 can include instructions to carry out or supplement the processes described above, and can include secure information also. Thus, for example, the expansion memory 574 can be provide as a security module for the mobile computing device 550, and can be programmed with instructions that permit secure use of the mobile computing device 550. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 564, the expansion memory 574, or memory on the processor 552. In some implementations, the computer program product can be received in a propagated signal, for example, over the transceiver 568 or the external interface 562.

The mobile computing device 550 can communicate wirelessly through the communication interface 566, which can include digital signal processing circuitry where necessary. The communication interface 566 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 568 using a radio-frequency. In addition, short-range communication can occur, such as using a Bluetooth, WiFi, or other such transceiver (not shown). In addition, a GPS (Global Positioning System) receiver module 570 can provide additional navigation- and location-related wireless data to the mobile computing device 550, which can be used as appropriate by applications running on the mobile computing device 550.

The mobile computing device 550 can also communicate audibly using an audio codec 560, which can receive spoken information from a user and convert it to usable digital information. The audio codec 560 can likewise generate audible sound for a user, such as through a speaker, e.g., in a handset of the mobile computing device 550. Such sound can include sound from voice telephone calls, can include recorded sound (e.g., voice messages, music files, etc.) and can also include sound generated by applications operating on the mobile computing device 550.

The mobile computing device 550 can be implemented in a number of different forms, as shown in the figure. For example, it can be implemented as a cellular telephone 580. It can also be implemented as part of a smart-phone 582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., sensory feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

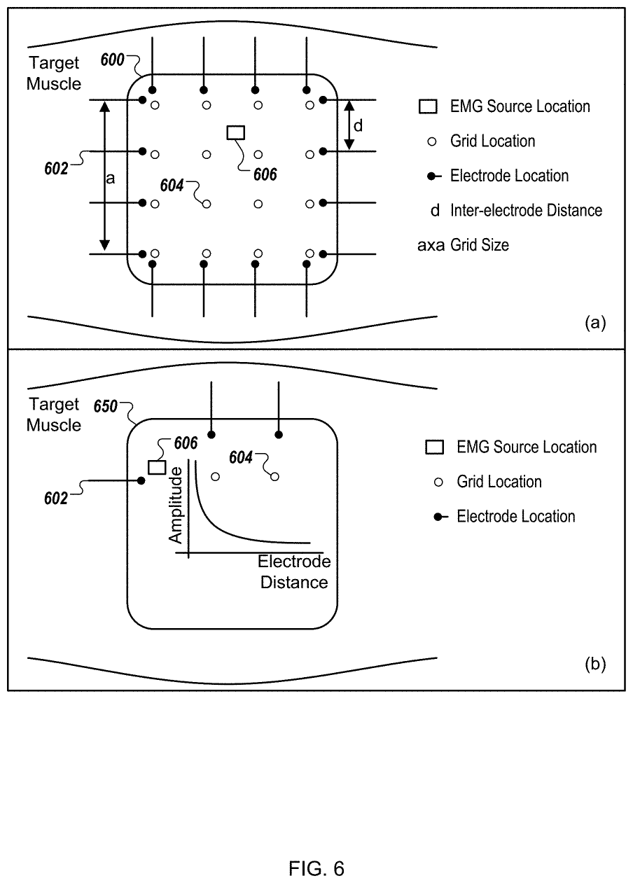

FIG. 6 shows two examples of sensor hardware 600 and 650. The sensor hardware 600 and 650 can be used combination with the other devices, apparatus, systems, and methods described throughout this document, for example, being used in the harness 102 for the treatment of dystonic symptoms. Other uses and/or implementations of the sensor hardware 600 and 650 are also possible.

The hardware 600 and 650 includes a plurality of electrodes 602 spaced d apart from each other and a grid of sensors 604 in a grid of size a×a secured on a patch that may, for example, include an adhesive to secure the hardware 600 and 650 to a subject's skin.

In the hardware 600 and 650, EMG signal from a patient, such as a patient's arm and hand, can be sensed. In one example, to detect the dystonia of a pianist during practice, the sensors can be attached onto the pianist's arms at various locations and data can be collected while he or she is playing. The collected data can be compared with the EMG data from conventional bulky EMG sensors. By comparison, the hardware 600 will be able to define the best measurement location.

The various parameters (e.g., d and a) of the hardware 600 and 650 can be adjusted based on the capabilities of the various components. For example, factors such as the sensor's 604 conductivity, impedance, sensor geometry, substrates and manufacturing procedure can affect the sensitivity and performance of the sensors. Substrates made by various materials, thicknesses, Young's modulus can be systematically analyzed to select parameters to improve the sensitivity of the sensors. Various corona discharging parameters such as voltage, needle-electrode distance, etc. can be analyzed to select parameters of the manufacturing process or placement of the sensors, their sensitivity, data repeatability and reliability. In many cases, large size skin-like sensors can be manufactured at low cost, allowing the capability to map the muscle movement of the whole limb (e.g., arm, leg) at real time. This can allow in-situ mapping of muscle movement of, e.g., a pianist to identify the muscle behavior before and after dystonia to offer better settings the technology to prevent dystonia. Hardware 600 shows a square sensor patch (other shapes are also possible) with an example EMG source 606 that is detected by the hardware 600. The sensor array 604 is designed such that the EMG source 606 can be detected and localized based on comparison of the differing signals detected by the sensor array 604. For instance, the location of the EMG source 606 can be determined from signals detected by adjacent sensors (i.e., EMG source 606 example location between two grid points (similar to a pixel of a digital image)) and a relief feedback can be given accordingly to ease the dystonic muscle. Hence, a control algorithm can be used, based on, for example, an analysis of EMG images in space and time, to estimate the source location of dystonic EMG signal(s), and provide relief feedback to the muscle using a vibrotactor (e.g. tactor array 108). The control algorithm can also be used to notify the user about possible onset of dystonia allowing for natural correction mechanisms. Further, machine learning techniques can be used to generate one or more mathematical models using the EMG data for each user to predict the possible onset of focal dystonia. The hardware 650 shows a square sensor patch with an example EMG source 606 that can be detected by the hardware 650. The amplitude of the EMG signal can decay with increasing distance. Different levels of dystonia and the corresponding EMG signal characteristics can be used to set parameters such as the array sensitivity and grid area of the sensors 604 for accurately mapping the target muscle. Patterns which can match well with the dystonia area are designed and tested for improved monitoring capability manufacturing ease.

What is claimed is:

1. A system for the treatment of dystonic symptoms, the system comprising:
one or more surface-electromyogram (EMG) sensors configured to:
attach to a patient;
sense surface-EMG data indicative of muscle movement of the patient; and
transmit, to a controller, the surface-EMG data;
a controller comprising computer-memory and one or more processors, the computer memory storing instructions that, when executed by the processor, cause the controller to:
receive, from the surface-EMG sensors, the surface-EMG data;
identify at least one parameter of the surface-EMG data;
compare the parameter to a corresponding dystonia-threshold-value;
responsive to a determination that the parameter is greater than the corresponding threshold-value:
issue a visual-engagement command to a visual unit; and
issue a tactile-engagement command to a tactile unit;
the visual unit configured to:
receive the visual-engagement command; and
responsive to receiving the visual-engagement command, generating a visual stimulation for the patient;
the tactile unit configured to:
receive the tactile-engagement command; and
responsive to receiving the tactile-engagement command; generating a tactile stimulation for the patient.

2. The system of claim 1, wherein the system further comprises a harness shaped to be wearable by the patient, the harness further being configured to secure the surface-EMG sensors, the controller, the visual unit, and the tactile unit.

3. The system of claim 2, wherein the harness comprises a sleeve wearable on the patient's arm.

4. The system of claim 2, wherein the harness comprises a glove wearable on the patient's hand.

5. The system of claim 1, wherein the surface-EMG sensors comprise graphine and a silver electrode.

6. The system of claim 1, wherein the surface-EMG sensors are configured to be worn on the patient's forearm.

7. The system of claim 6, wherein the surface-EMG sensors are configured to sense surface-EMG data indicative of muscle movements in at least one of the group consisting of Flexor Carpi Ulnaris, Palmaris Longus, Extensor Digitorum, and Extensor Carpi Radialis.

8. The system of claim 6, wherein the wherein the surface-EMG sensors are configured to sense surface-EMG data indicative of muscle movements in each of the group consisting of Flexor Carpi Ulnaris, Palmaris Longus, Extensor Digitorum, and Extensor Carpi Radialis.

9. The system of claim 1, wherein the at least one parameter is a dystonic signal present in the surface-EMG data when the patient is experiencing dystonic symptoms.

10. The system of claim 1, wherein the system is configured to deliver the visual stimulation to the patient when the patient is experiencing dystonic symptoms.

11. The system of claim 1, wherein the system is configured to deliver the tactile stimulation to the patient when the patient is experiencing dystonic symptoms.

12. The system of claim 1, the system further comprising at least one battery in energetic communication with at least the controller.

13. The system of claim 1, wherein the visual stimulation comprises illumination of a light-emitting diode (LED).

14. The system of claim 1, wherein the tactile stimulation comprises engagement of a tactor that generates a vibration on the patient's skin.

15. The system in claim 1, wherein the parameter is a first value in volts and wherein the corresponding dystonia-threshold-value is a second value in volts.

16. A non-transitory, computer-readable media storing instructions that, when executed by a processor, cause a controller of the processor to:
receive, from a surface-EMG sensors, surface-EMG data indicative of muscle movement of a patient;
identify at least one parameter of the surface-EMG data;
compare the parameter to a corresponding dystonia-threshold-value;
responsive to a determination that the parameter is greater than the corresponding threshold-value:
issue a visual-engagement command to a visual unit to generate a visual stimulation for the patient; and
issue a tactile-engagement command to a tactile unit to a tactile unit to generate a tactile stimulation for the patient.

17. The media of claim 15, wherein the at least one parameter is a dystonic signal present in the surface-EMG data when the patient is experiencing dystonic symptoms.

18. The media of claim 15, wherein the media is configured to cause delivery of the visual stimulation to the patient when the patient is experiencing dystonic symptoms.

19. The media of claim 15, wherein the media is configured to cause delivery of the tactile stimulation to the patient when the patient is experiencing dystonic symptoms.

20. The media of claim 15, wherein the parameter is a first value in volts and wherein the corresponding dystonia-threshold-value is a second value in volts.
